(19) United States
(12) Patent Application Publication
(10) Pub. No.: US 2023/0349004 A1
(43) Pub. Date: Nov. 2, 2023

(54) METHOD FOR DETECTING VIRULENT STRAINS OF VENTURIA INAEQUALIS

(71) Applicants: Institut National De Recherche Pour L'agriculture, L'alimentation Et L’environnement, Paris (FR); Insititut National Supérieur des Sciences Agronomiques, Agroalimentaires, Horticoles et du Paysage, Rennes (FR); Universite D'Angers, Angers (FR)

(72) Inventors: Bruno Le Cam, La Meignanne (FR); Mélanie Sannier, Montreuil Sur Maine (FR); Christophe Lemaire, Vair Sur Loire (FR); Jérôme Collemare, Heerde (NL)

(21) Appl. No.: 18/025,734

(22) PCT Filed: Sep. 9, 2021
(86) PCT No.: PCT/EP2021/074818

§ 371 Date: Mar. 10, 2023

(30) Foreign Application Priority Data
Sep. 10, 2020 (FR) 20 09176

(51) Int. Cl. C12Q 1/6895; C12Q 1/6851

(52) U.S. Cl. CPC C12Q 1/6895; C12Q 1/6851; C12Q 2600/156

(57) ABSTRACT

The present invention relates to a method for detecting the presence of a virulent strain of Venturia inaequalis in a sample. Said method is based on identifying specific markers associated with virulent strains of Venturia inaequalis.

Said detection method has a plurality of advantageous applications, such as determining geographical areas where strains are virulent, enabling, in particular, evaluation of the benefit of planting an orchard in said area with varieties carrying the corresponding resistance gene, evaluation of whether fungicidal treatment in an orchard is necessary, thus avoiding systematic preventive treatment, or evaluation of the health quality of an apple plant comprising the resistance gene Rvi6.

### TECHNICAL FIELD

The present invention relates to the detection of virulent strains of Venturia inaequalis.

### PRIOR ART

Apple scab is one of the main fungal diseases of apple trees. The pathogen responsible for apple scab is an ascomycete fungus called Venturia inaequalis. Apple scab is characterized by the appearance of black or brown circular spots on the surface of leaves, buds, fruits and/or wood of infected fruit trees. Fruits and leaves are particularly sensitive to Venturia inaequalis.

Scab is favored by a humid climate in the spring at the time of bud break. The period of primary contamination related to ascospore dispersion begins in spring. Primary contaminations will lead to the development of sporulating symptoms on young leaves and young fruits. In rainy periods during spring and summer, spores dispersed by water drops and wind will initiate secondary contamination.

In orchards, scab does not cause the death of the affected apple tree but can significantly reduce the productivity of the affected tree and especially the quality of fruits since no scab lesion is tolerated on fruits in the distribution circuits. To protect the apple orchards thereof, producers use chemical control. Thus, whether in conventional or organic agriculture, the control of apple scab requires an average of 25 fungicidal treatments per year or even more during rainy years.

To date, genetic control is essentially based on the use of a single resistant gene, the gene Rvi6 from the ornamental species Malus floribunda that breeders have inserted into some varieties. However, in certain parts of Europe, the emergence of virulent strains of Venturia inaequalis has entailed a loss of efficacy of such resistance.

To date, there are no ways to detect the presence of Venturia inaequalis strains which are virulent against the resistance gene Rvi6. As a result, as a precautionary principle, preventive fungicidal treatments are systematically applied during the risk period in spring, for protecting the varieties carrying the gene Rvi6. Similarly, certain growers are reluctant to plant varieties bearing Rvi6 resistance for lack of information on the presence of virulent populations in the environment thereof.

There is thus a real need to provide a method for detecting virulent strains of Venturia inaequalis, which is preferably quick and easy to implement.

### DETAILED DESCRIPTION

The inventors have succeeded in identifying and sequencing the avirulence gene AvrRvi6 of the fungus V. inaequalis which interacts with the resistance gene Rvi6. It is the nucleotide modifications at said locus (mutations such as substitution(s), insertion(s) and/or deletion(s) which are responsible for the gain in virulence of the mutated strains. The Inventors have indeed identified a genetic polymorphism at the AvrRvi6 locus, and at the promoter thereof, within 120 strains of Venturia inaequalis. On the basis of such polymorphism, the Inventors have developed a method for detecting the presence of virulent strains of Venturia inaequalis, which is based on the specific detection of the alleles and/or the promoter of the gene AvrRvi6 imparting a virulent character to a strain.

The detection method according to the invention has the advantage of being simple to implement and quick, with a result which can be obtained in a few days. The detection method according to the invention is furthermore sensitive and precise, in particular due to the use of technologies such as quantitative PCR (Polymerase Chain reaction), in particular isothermal quantitative PCR, LAMP (Loop-mediated Amplification) and/or Qprobe whether or not combined with the LAMP technique.

The detection method according to the invention thereby represents an essential decision-making support tool for determining whether virulence is present at different scales (e.g., within an orchard, a farm, a nursery, a country or even a continent). The method according to the invention can also be used for greatly contributing to a reduced use of fungicides, (1) by making it possible to apply treatments only in the presence of the virulent population, thereby avoiding systematic preventive treatment and (2) by encouraging growers to plant varieties carrying the resistance gene Rvi6.

The detection method according to the invention can further be used for verifying the quality of plants and e.g. for guaranteeing that a plant comprising the resistance gene Rvi6 is free of any contamination by a strain of Venturia inaequalis virulent towards the gene Rvi6.

A first subject matter of the invention relates to a method for detecting the presence of a virulent strain of Venturia inaequalis in a sample, comprising:
a) providing a sample likely to comprise Venturia inaequalis DNA,
b) determining whether the DNA present in the sample contains at least one virulence polymorphism,
c) If at least one virulence polymorphism is detected during step b), deducing therefrom that the sample provided during step a) comprises a virulent strain of Venturia inaequalis.

The virulence polymorphism is preferentially an allele comprising or consisting of a sequence selected from the group consisting of SEQ ID NO: 1, SEQ ID NO: 2, SEQ ID NO: 3, SEQ ID NO: 4, SEQ ID NO: 5 and SEQ ID NO: 6 and/or a promoter comprising or consisting of SEQ ID NO: 7 or SEQ ID NO: 8.

Step a) of the method as defined above preferentially comprises the following steps:
(i) providing a sample likely to comprise a strain of Venturia inaequalis,
(ii) extracting the DNA from the sample provided during step (i), for obtaining a sample likely to comprise Venturia inaequalis DNA.

Step b) of the method as defined above preferentially comprises determining whether the DNA comprises at least one virulence marker selected from the group consisting of:
a fragment of sequence SEQ ID NO: 1 comprising nucleotides 155 to 165 of said sequence SEQ ID NO: 1,
a fragment of sequence SEQ ID NO: 2 comprising nucleotide 62 of said sequence SEQ ID NO: 2,
a fragment of sequence SEQ ID NO: 3 comprising nucleotide 160 of said sequence SEQ ID NO: 3,
a fragment of sequence SEQ ID NO: 4 comprising nucleotide 192 of said sequence SEQ ID NO: 4,
a fragment of sequence SEQ ID NO: 5 comprising nucleotide 201 of said sequence

SEQ ID NO: 5,
a fragment of sequence SEQ ID NO: 6 comprising nucleotide 304 of said sequence SEQ ID NO: 6,
a fragment of sequence SEQ ID NO: 7 comprising nucleotide 189, nucleotides 304 to 316 and/or nucleotides 480 to 490 of said sequence SEQ ID NO: 7, and
a fragment of sequence SEQ ID NO: 8 comprising nucleotide 572 of said sequence SEQ ID NO: 8, nucleotide 706 of said sequence SEQ ID NO: 8, nucleotides 548 to 557 of said sequence SEQ ID NO: 8 and/or 15 consecutive nucleotides comprised between nucleotides 139 to 420 of said sequence SEQ ID NO: 8.

Another subject matter of the invention relates to a method for the preventive and/or curative treatment of apple trees, comprising the resistance gene Rvi6, enabling reducing the frequency and/or the quantity of phytosanitary product applied against Venturia inaequalis, comprising:
implementing steps a) to c) of the detection method as defined above,
applying preventive and/or curative treatment if the sample provided during step a) includes a virulent strain of Venturia inaequalis.

Another subject matter of the invention relates to a method for evaluating the quality of an apple plant, comprising the following steps:
implementing steps a) to c) of the detection method as defined above, the sample provided during step a) being sampled from the air around said plant and/or from all or part of said plant,
if no virulence polymorphism is detected during step b), deducing therefrom that the plant is not contaminated with a virulent Venturia inaequalis strain comprising the virulence polymorphism sought during step b).

Another subject matter of the invention relates to a method for mapping the virulence of Venturia inaequalis comprising the following steps:
Implementing steps a) to c) of the detection method as defined above, from at least two samples sampled from different geographical areas,
deducing therefrom that the geographical area from which comes a sample comprising a virulent strain of Venturia inaequalis, is a virulence area and that the geographical area from which comes a sample not comprising a virulent strain of Venturia inaequalis, is a virulence-free area.

Another subject matter of the invention relates to a virulence marker comprising or consisting of:
a fragment of sequence SEQ ID NO: 1 comprising nucleotides 155 to 165 of said sequence SEQ ID NO: 1,
a fragment of sequence SEQ ID NO: 2 comprising nucleotide 62 of said sequence SEQ ID NO: 2,
a fragment of sequence SEQ ID NO: 3 comprising nucleotide 160 of said sequence SEQ ID NO: 3,
a fragment of sequence SEQ ID NO: 4 comprising nucleotide 192 of said sequence SEQ ID NO: 4,
a fragment of sequence SEQ ID NO: 5 comprising nucleotide 201 of said sequence SEQ ID NO: 5,
a fragment of sequence SEQ ID NO: 6 comprising nucleotide 304 of said sequence SEQ ID NO: 6,
a fragment of sequence SEQ ID NO: 7 comprising nucleotide 189, nucleotides 304 to 316 and/or nucleotides 480 to 490 of said sequence SEQ ID NO: 7, or
a fragment of sequence SEQ ID NO: 8 comprising nucleotide 572 of said sequence SEQ ID NO: 8, nucleotide 706 of said sequence SEQ ID NO: 8, nucleotides 548 to 557 of said sequence SEQ ID NO: 8 and/or 15 consecutive nucleotides comprised between nucleotides 139 to 420 of said sequence SEQ ID NO: 8, said fragment comprising at least 15 nucleotides.

Another subject matter of the invention relates to a combination of at least two virulence markers, said markers being as defined above.

Another subject matter of the invention is a kit for detecting the presence of a virulent strain of Venturia inaequalis in a sample, said kit comprising means for amplifying a marker as defined above or the markers of the combination as defined above.

Another subject matter of the invention relates to the use of a virulence marker as defined above, of a combination of markers as defined above or of a kit as defined above, for detecting the presence of a virulent strain of Venturia inaequalis or for determining whether a strain of Venturia inaequalis is virulent or avirulent, in particular for determining geographical areas of virulence, for evaluating whether fungicidal treatment is needed, for evaluating the quality of an apple plant and/or apple tree fruits and/or for evaluating whether virulence is present over a given territory, so as to support the decision-making on whether or not to plant apple tree varieties carrying the resistance gene Rvi6.

### Venturia inaequalis

The term “apple scab” refers herein to a cryptogamic disease caused by the fungus Venturia inaequalis.

Apple scab affects apple trees of the genus Malus.

However, apple trees of the genus Malus comprising the resistance gene Rvi6 are resistant to so-called avirulent avrRvi6 strains of Venturia inaequalis.

The fungus Venturia inaequalis indeed comprises an avirulence gene AvrRvi6.

In an avirulent strain of Venturia inaequalis, the protein encoded by the avirulence gene AvrRvi6 is recognized, either directly or indirectly, by the protein encoded by the resistance gene Rvi6 expressed by the apple tree, thereby initiating a rapid defense reaction of the apple tree, which prevents the further development of the pathogen. The apple tree is then said to be “resistant”.

“Venturia inaequalis strain” refers herein to a single individual which can be distinguished by the genotype thereof.

The strain of Venturia inaequalis can be in the form of spores or of mycelium.

The strain of Venturia inaequalis is usually present on an infected plant in the form of lesions resulting from the presence of spores and of mycelium.

The terms “avirulent strain of Venturia inaequalis” or “strain of Venturia inaequalis having an avirulent character” refer herein to a strain of Venturia inaequalis comprising an avirulence gene AvrRvi6 encoding a protein recognized, either directly or indirectly, by the protein encoded by the resistance gene Rvi6 of the apple tree.

The terms “virulent strain of Venturia inaequalis” or “strain of Venturia inaequalis having a virulent character” refer herein to a strain of Venturia inaequalis which is not recognized as a pathogenic agent by an apple tree comprising the resistance gene Rvi6; such a strain is thus responsible for disease in the varieties carrying the gene Rvi6.

A virulent strain of Venturia inaequalis can contain at least one mutation in the

avirulence gene AvrRvi6 and/or in the avirulence gene promoter AvrRvi6, thereby resulting in the absence of expression of the protein and/or in a protein which is no longer recognized by the protein encoded by the resistance gene Rvi6.

The term “locus” refers to a fixed position of a gene or of a genetic marker on a chromosome.

The term “allele” refers herein to a variant of a gene located at a given locus.

The expression “polymorphism imparting a virulent (or an avirulent) character” or “virulence (or avirulence) polymorphism” refers herein to an allele and/or a promoter sequence imparting a virulent (or an avirulent) character on a strain of Venturia inaequalis.

An allele imparting a virulent character on a strain of Venturia inaequalis at the locus AvRvi6, comprises or consists e.g. of a sequence selected from the group consisting of SEQ ID NO: 1, SEQ ID NO: 2, SEQ ID NO: 3, SEQ ID NO: 4, SEQ ID NO: 5 and SEQ ID NO: 6.

SEQ ID NO: 1 comprises a substitution at nucleotide 139, in particular by the presence of nucleotide A instead of nucleotide G, and a deletion of nucleotides 155 to 309, e.g. with respect to the sequence SEQ ID NO: 15.

SEQ ID NO: 2 is characterized by the presence of a substitution at nucleotide 62, in particular by the presence of nucleotide C instead of nucleotide G, by a substitution at nucleotide 139, in particular by the presence of nucleotide A instead of nucleotide G, by a substitution at nucleotide 237, in particular by the presence of nucleotide C instead of nucleotide T and of a substitution at nucleotide 279, in particular by the presence of nucleotide A instead of T.

SEQ ID NO: 3 is characterized by the presence of a substitution at nucleotide 139, in particular by the presence of nucleotide A instead of nucleotide G, a substitution at nucleotide 160, in particular by the presence of nucleotide C instead of nucleotide T and by the presence of a substitution at nucleotide 279, in particular by the presence of nucleotide A instead of nucleotide T.

SEQ ID NO: 4 is characterized by the presence of a substitution at nucleotide 139, in particular by the presence of nucleotide A instead of nucleotide G, by a substitution at nucleotide 192, in particular by the presence of nucleotide A instead of C, by a substitution at nucleotide 219, in particular by the presence of nucleotide G instead of nucleotide A and of a substitution at nucleotide 279, in particular by the presence of nucleotide A instead of nucleotide T.

SEQ ID NO: 5 is characterized by the presence of a substitution at nucleotide 139, in particular by the presence of nucleotide A instead of nucleotide G, of a substitution at nucleotide 201, in particular by the presence of nucleotide C instead of nucleotide T, and of a substitution at nucleotide 279, in particular by the presence of nucleotide A instead of nucleotide T.

SEQ ID NO: 6 is characterized by the presence of a substitution at nucleotide 304, in particular by the presence of nucleotide G instead of nucleotide T.

An example of an allele imparting an avirulent character on a strain of Venturia inaequalis at the locus AvRvi6 is an allele comprising or consisting of the sequence SEQ ID NO: 15.

A promoter imparting a virulent character to a strain of Venturia inaequalis at the locus AvRvi6 comprises or consists e.g. of SEQ ID NO: 7 or SEQ ID NO: 8.

SEQ ID NO: 7 is characterized by the presence of a substitution at nucleotide 189, in particular by the presence of nucleotide A instead of nucleotide G, by the presence of three deletions of nucleotides 309 to 311, in particular by the deletion of nucleotides A, T and A and by inserting 5 nucleotides in position 484, in particular nucleotides G, A, A, T and A.

SEQ ID NO: 8 is characterized by the presence of an insertion of 281 nucleotides at nucleotide 139, a deletion of nucleotides 552 to 555, a substitution at nucleotide 572, in particular by the presence of nucleotide A instead of C and a substitution of nucleotide 706, in particular by the presence of nucleotide G instead of A.

An example of a promoter imparting an avirulent character on a strain of Venturia inaequalis at the locus AvRvi6 is an allele comprising or consisting of SEQ ID NO: 14.

### Virulence Marker, Marker for the Presence of Venturia inaequalis, Combination of Markers

A further subject matter of the present invention is a virulence marker, a Venturia inaequalis marker and/or a combination of at least two virulence markers and, optionally, at least one marker for the presence of V. inaequalis.

“Virulence marker” refers herein to a polynucleotide the sequence of which corresponds to a region present at the avirulence locus of a virulent strain of Venturia inaequalis, yet different from the avirulence locus of avirulent strains of Venturia inaequalis. A virulence marker comprises e.g. at least one mutation of a nucleotide (substitution, 5 addition and/or deletion) with respect to the sequence present in an avirulent strain of Venturia inaequalis.

Table 1 shows the single nucleotide polymorphisms (SNPs) and/or deletions present in the alleles comprising or consisting of SEQ ID NO: 1 to SEQ ID NO: 6. The position of the SNP or of deletion is indicated with respect to SEQ ID NO: 15.

TABLE 1

| Allele | SNP | SNP | SNP | SNP | SNP | SNP | SNP | SNP | SNP | |
|---|---|---|---|---|---|---|---|---|---|---|
| (sequence | 62 | 139 | 160 | 192 | 201 | 219 | 237 | 279 | 304 | Deletion |
| SEQ ID NO:) | C | A | C | A | C | G | C | A | G | 155-309 |
| 1 | | x | | | | | | | | x |
| 2 | x | x | | | | | x | x | | |
| 3 | | x | x | | | | | x | | |
| 4 | | x | | x | | x | | x | | |
| 5 | | x | | | x | | | x | | |
| 6 | | | | | | | | | x | |

The present invention thus relates to a virulence marker comprising or consisting of:
a fragment of sequence SEQ ID NO: 1 comprising nucleotides 155 to 165 of said sequence SEQ ID NO: 1, preferentially comprising nucleotides 155 to 180 of said sequence SEQ ID NO: 1, wherein said marker is in particular specific for the allele of sequence SEQ ID NO: 1,
a fragment of sequence SEQ ID NO: 2 comprising nucleotide 62 of said sequence, said marker being in particular specific for the allele of sequence SEQ ID NO: 2,
a fragment of sequence SEQ ID NO: 3 comprising nucleotide 160 of said sequence, said marker being in particular specific for the allele of sequence SEQ ID NO: 3,
a fragment of sequence SEQ ID NO: 4 comprising nucleotide 192 of said sequence, said marker being in particular specific for the allele of sequence SEQ ID NO: 4,
a fragment of sequence SEQ ID NO: 5 comprising nucleotide 201 of said sequence, said marker being in particular specific for the allele of sequence SEQ ID NO: 5,
a fragment of sequence SEQ ID NO: 6 comprising nucleotide 304 of said sequence, said marker being in particular specific for the allele of sequence SEQ ID NO: 6,
a fragment of sequence SEQ ID NO: 7 comprising nucleotides 189, nucleotides 304 to 316 and/or nucleotides 480 to 490 of said sequence SEQ ID NO: 7, said marker being in particular specific for the promoter of sequence SEQ ID NO: 7, or
a fragment of sequence SEQ ID NO: 8 comprising nucleotide 572 of said sequence SEQ ID NO: 8, nucleotide 706 of said sequence SEQ ID NO: 8, nucleotides 548 to 557 of said sequence SEQ ID NO: 8 and/or 15 consecutive nucleotides comprised between nucleotides 139 to 420 of said sequence SEQ ID NO: 8, said marker being in particular specific for the promoter of sequence SEQ ID NO: 8.

The subject matter of the present invention is e.g. a virulence marker as defined above comprising or consisting of:
a fragment of sequence SEQ ID NO: 1 comprising nucleotides 155 to 165 of said SEQ ID NO: 1, preferentially comprising nucleotides 155 to 180 of said sequence SEQ ID NO: 1,
a fragment of sequence SEQ ID NO: 2 comprising (i) nucleotide 62 and (ii), optionally, nucleotide 139, nucleotide 237 and/or nucleotide 279 of said sequence SEQ ID NO: 2,
a fragment of sequence SEQ ID NO: 3 comprising (i) nucleotide 160 and (ii), optionally, nucleotide 139 and/or nucleotide 279 of said sequence SEQ ID NO: 3,
a fragment of sequence SEQ ID NO: 4 comprising (i) nucleotide 192 and (ii), optionally, nucleotide 139, nucleotide 219 and/or nucleotide 279 of said sequence SEQ ID NO: 4,
a fragment of sequence SEQ ID NO: 5 comprising (i) nucleotide 201 and (ii), optionally, nucleotide 139 and/or nucleotide 279 of said sequence SEQ ID NO: 5,
a fragment of sequence SEQ ID NO: 6 comprising nucleotide 304 of said sequence SEQ ID NO: 6,
a fragment of sequence SEQ ID NO: 7 comprising nucleotides 189, nucleotides 304 to 316 and/or nucleotides 480 to 490 of said sequence SEQ ID NO: 7, or
a fragment of sequence SEQ ID NO: 8 comprising nucleotide 572 of said sequence SEQ ID NO: 8, nucleotide 706 of said sequence SEQ ID NO: 8, nucleotides 548 to 557 of said sequence SEQ ID NO: 8 and/or 15 consecutive nucleotides comprised between nucleotides 139 to 420 of said sequence SEQ ID NO: 8.

“Fragment” refers herein to a polynucleotide with a length of at least 5 nucleotides, preferentially at least 8 nucleotides, more preferentially at least 10 nucleotides, e.g. at least or at least 20 nucleotides. The fragment is preferentially a fragment with a size less than or equal to 50 nucleotides, preferentially less than or equal to 40 nucleotides, e.g. less than or equal to 30 nucleotides.

A fragment as defined above preferentially comprises at least 15 nucleotides.

“A fragment of SEQ ID NO: X” refers to a fragment consisting of consecutive nucleotides of a polynucleotide of SEQ ID NO: X.

Thereby, the subject matter of the present invention is e.g. a virulence marker comprising or consisting of:
a polynucleotide comprising or consisting of nucleotides 155 to 180 of sequence SEQ ID NO: 1,
a polynucleotide comprising or consisting of nucleotides 52 to 72 of sequence SEQ ID NO: 2,
a polynucleotide comprising or consisting of nucleotides 150 to 170 of sequence SEQ ID NO: 3,
a polynucleotide comprising or consisting of nucleotides 182 to 202 of sequence SEQ ID NO: 4,
a polynucleotide comprising or consisting of nucleotides 191 to 211 of sequence SEQ ID NO: 5,
a polynucleotide comprising or consisting of nucleotides 294 to 314 of sequence SEQ ID NO: 6,
a polynucleotide comprising or consisting of nucleotides 180 to 200, nucleotides 304 to 316 and/or nucleotides 480 to 490 of sequence SEQ ID NO: 7, or
a polynucleotide comprising or consisting of nucleotides 562 to 582, nucleotides 696 to 716, nucleotides 548 to 557 and/or 15 consecutive nucleotides between nucleotides 139 to 420 of sequence SEQ ID NO: 8.

“A marker of the presence of V. inaequalis”, refers herein to a polynucleotide the sequence of which corresponds to a region present in all the strains of Venturia inaequalis.

A marker for the presence of V. inaequalis which can be used is e.g. a polynucleotide comprising all or part of the sequence encoding actin of V. inaequalis, a polynucleotide comprising all or part of the sequence encoding an elongation factor of V. inaequalis or a polynucleotide comprising all or a portion of the housekeeping gene RPL4 of V. inaequalis.

A marker for the presence of a preferred V. inaequalis is a polynucleotide comprising all or a portion of the housekeeping gene RPL4 of V. inaequalis.

Thereby, the subject matter of the present invention is a marker for the presence of Venturia inaequalis comprising or consisting of SEQ ID NO: 9 or a fragment of SEQ ID NO: 9.

A marker for the presence of Venturia inaequalis is e.g. the polynucleotide corresponding to nucleotides 1243 to 1419 of SEQ ID NO: 9.

An example of a pair of primers for amplifying a marker of the presence of Venturia inaequalis, in particular the housekeeping gene RPL4, consists of the forward primer of sequence SEQ ID NO: 10 (TTCAATCTGCTCCGAGGT) and the reverse primer of sequence SEQ ID NO: 11 (CTAATGCCTTTGGCTTCTCG).

A further subject matter of the present invention is a combination of at least two virulence markers and, optionally, at least one marker for the presence of V. inaequalis.

Virulence markers and/or the marker for the presence of V. inaequalis of said combination are in particular as defined above.

The combination of at least two virulence markers and, optionally, at least one marker for the presence of V. inaequalis can comprise e.g.:
at least 2 virulence markers, preferentially at least 4 virulence markers, preferentially at least 6 virulence markers, e.g. 6, 7, 8 or at least 8 virulence markers and
optionally, one or at least one marker for the presence of V. inaequalis.

A preferential combination of at least two virulence markers and, optionally, at least one marker for the presence of V. inaequalis can include:
8 virulence markers, e.g.:
   a polynucleotide comprising or consisting of nucleotides 155 to 180 of sequence SEQ ID NO: 1,
   a polynucleotide comprising or consisting of nucleotides 52 to 72 of sequence SEQ ID NO: 2,
   a polynucleotide comprising or consisting of nucleotides 150 to 170 of sequence SEQ ID NO: 3,
   a polynucleotide comprising or consisting of nucleotides 182 to 202 of sequence SEQ ID NO: 4,
   a polynucleotide comprising or consisting of nucleotides 191 to 211 of sequence SEQ ID NO: 5,
   a polynucleotide comprising or consisting of nucleotides 294 to 314 of sequence SEQ ID NO: 6,
   a polynucleotide comprising or consisting of nucleotides 180 to 200, nucleotides 304 to 316 and/or nucleotides 480 to 490 of sequence SEQ ID NO: 7, and
   a polynucleotide comprising or consisting of nucleotides 562 to 582, nucleotides 696 to 716, nucleotides 548 to 557 and/or 15 consecutive nucleotides between nucleotides 139 to 420 of sequence SEQ ID NO: 8, and
optionally, a marker for the presence of V. inaequalis, e.g. a marker for the presence of Venturia inaequalis comprising or consisting of SEQ ID NO: 9 or a fragment of SEQ ID NO: 9 is as defined above.

### Method for Detecting the Presence of a Virulent Strain of Venturia inaequalis

The present invention particularly relates to a method for detecting the presence of a virulent strain of Venturia inaequalis, in particular against an apple tree comprising a resistance gene Rvi6, in a sample, comprising:
a) providing a sample likely to comprise Venturia inaequalis DNA,
b) determining whether the DNA in the sample contains at least one virulence polymorphism and, optionally, at least one marker for the presence of V. inaequalis,
c) if at least one virulence polymorphism is detected during step b), deducing therefrom that the sample provided during step a) comprises a virulent strain of Venturia inaequalis.

### Step a)

Step a) comprises or consists of providing a sample likely to comprise Venturia inaequalis DNA.

The expression “likely to comprise DNA” means that the starting sample may not contain a strain of Venturia inaequalis, in which case no Venturia inaequalis DNA will be present in the sample.

The presence of Venturia inaequalis in the sample can be verified during step b) by determining the presence of at least one marker of the presence of V. inaequalis.

In a preferred embodiment, step a) of providing a sample likely to comprise Venturia inaequalis DNA comprises the following steps:
(i) providing a sample likely to comprise at least one strain of Venturia inaequalis,
(ii) extracting the DNA from the sample provided during step (i), for obtaining a sample likely to comprise Venturia inaequalis DNA.

The sample likely to comprise at least one strain of Venturia inaequalis can be sampled by any method well known to a person skilled in the art.

The sample likely to comprise at least one strain of Venturia inaequalis can be sampled from the air, e.g. by using one or a plurality of spore traps, in particular placed next to an apple tree, e.g. in the center of an orchard, from a nursery or spread over the surface of an orchard or on the ground and/or can be sampled from apple leaves and/or fruits. DNA e.g. can be extracted from tissues exhibiting symptoms, such as fruit and/or leaf tissues.

The strain or strains of Venturia inaequalis are preferentially present in the sample as spores and/or mycelium, e.g. on or in plant tissues (leaves and/or fruits).

The sample likely to comprise at least one strain of Venturia inaequalis thus preferentially comprises spores of Venturia inaequalis and/or apple tissue(s).

DNA is extracted by any appropriate method well known to a person skilled in the art e.g. by using a commercial DNA extraction kit or a manual extraction method, such as DNA extraction with CTAB (hexadecyltrimethylammonium bromide).

### Step b)

Step b) comprises or consists of determining whether the DNA present in the sample comprises at least one virulence polymorphism and, optionally, a marker for the presence of V. inaequalis.

The virulence polymorphism is preferentially an allele comprising or consisting of a sequence selected from the group consisting of SEQ ID NO: 1, SEQ ID NO: 2, SEQ ID NO: 3, SEQ ID NO: 4, SEQ ID NO: 5 and SEQ ID NO: 6 or a promoter comprising or consisting of SEQ ID NO: 7 or SEQ ID NO: 8.

In a preferred embodiment, step b) thereby comprises determining whether the DNA present in the sample comprises an allele comprising or consisting of sequence SEQ ID NO: 1, an allele comprising or consisting of sequence SEQ ID NO: 2, an allele comprising or consisting of sequence SEQ ID NO: 3, an allele comprising or consisting of sequence SEQ ID NO: 4, an allele comprising or consisting of sequence SEQ ID NO: 5, an allele comprising or consisting of sequence SEQ ID NO: 6, a promoter comprising or consisting of sequence SEQ ID NO: 7 and/or a promoter comprising or consisting of sequence SEQ ID NO: 8.

In a preferred embodiment, the presence of at least one virulence polymorphism in said DNA is determined by the presence of at least one virulence marker, in particular a virulence marker as defined above or a combination of virulence markers as defined above.

Preferentially, step b) thereby comprises determining whether said DNA comprises at least one virulence marker, in particular a virulence marker as defined above or a combination of virulence markers as defined above.

The method according to the invention thereby advantageously allows specifically detecting an allele or a promoter imparting a virulent character.

Preferentially, step b) comprises or consists in determining whether the DNA present in the sample comprises at least one virulence polymorphism and, optionally, a marker for the presence of V. inaequalis, the presence of at least one virulence polymorphism in said DNA being determined by the presence of at least one virulence marker as defined above, preferentially selected from the group consisting of:
a fragment of sequence SEQ ID NO: 1 comprising nucleotides 155 to 165 of said sequence SEQ ID NO: 1,
a fragment of sequence SEQ ID NO: 2 comprising nucleotide 62 of said sequence SEQ ID NO: 2,
a fragment of sequence SEQ ID NO: 3 comprising nucleotide 160 of said sequence SEQ ID NO: 3,
a fragment of sequence SEQ ID NO: 4 comprising nucleotide 192 of said sequence SEQ ID NO: 4,
a fragment of sequence SEQ ID NO: 5 comprising nucleotide 201 of said sequence SEQ ID NO: 5,
a fragment of sequence SEQ ID NO: 6 comprising nucleotide 304 of said sequence SEQ ID NO: 6,
a fragment of sequence SEQ ID NO: 7 comprising nucleotide 189, nucleotides 304 to 316 and/or nucleotides 480 to 490 of said sequence SEQ ID NO: 7, and
a fragment of sequence SEQ ID NO: 8 comprising nucleotide 572 of said sequence SEQ ID NO: 8, nucleotide 706 of said sequence SEQ ID NO: 8, nucleotides 548 to 557 of said sequence SEQ ID NO: 8 and/or 15 consecutive nucleotides comprised between nucleotides 139 to 420 of said sequence SEQ ID NO: 8,
   or by the presence of a combination of virulence markers as defined above.

Step b) as defined above preferentially comprises a step of checking the presence of DNA of a strain of Venturia inaequalis.

Such step of checking for the presence of DNA of a strain of Venturia inaequalis comprises e.g. determining whether the DNA present in the sample is Venturia inaequalis DNA.

The step of checking for the presence of DNA from a strain of Venturia inaequalis strain comprises e.g. determining whether a sequence common to virulent and avirulent strains is present in the sample, e.g. the sequence of a housekeeping gene RPL4.

The marker of the presence of V. inaequalis is thus used as a control marker in the detection method defined hereinabove.

The marker of the presence of V. inaequalis is e.g. a polynucleotide comprising all or part of the sequence of the gene RPL4 of V. inaequalis, e.g. comprising all or part of sequence SEQ ID NO: 9.

In order to determine whether the DNA present in the sample comprises at least one virulence polymorphism, any technique well known to a person skilled in the art can be used, such as the sequencing of all or part of the promoter sequence of the avirulence gene AvrRvi6 and/or the sequence of the allele present at the locus AvRvi6 and/or a DNA amplification technique, preferentially isothermal amplification.

Examples of DNA amplification, but not limited to, include PCR, quantitative PCR, isothermal quantitative PCR, LAMP (Loop-mediated Amplification) and/or Qprobe whether combined or not with the LAMP technique, RPA (Recombinase Polymerase Amplification) technique.

The LAMP method is an isothermal amplification method which has a very high level of specificity, linked to the use of 6 primers and is generally very fast. The detection of amplification by the LAMP method can be carried out by electrophoresis, turbidimetry, electrochemistry or colorimetry.

The RPA method is an isothermal amplification method carried out at low temperature. The detection of amplification by the RPA method is often carried out by fluorescence, using e.g. fluorescent probes or intercalating agents.

When a DNA amplification technique is used, at least one pair of primers apt to amplify a virulence marker as defined above and, optionally, at least one pair of primers apt to amplify a marker for the presence of V. inaequalis as defined above, is/are used.

The pair of primers apt to amplify a virulence marker can be used e.g. for obtaining:
a fragment amplified from the DNA of a virulent strain, but not from the DNA of an avirulent strain,
a fragment amplified from the DNA of an avirulent strain, but not from the DNA of a virulent strain, or
a fragment amplified from the DNA of a virulent strain with a size different from the DNA amplified from the DNA of an avirulent strain.

The forward primer and/or the reverse primer can e.g. hybridize at a virulence marker, in particular when the polymorphism at said marker results from one or a plurality of substitutions and/or deletions and/or insertions of nucleotide.

Alternatively, the forward primer and/or the reverse primer can hybridize around a virulence marker, in particular when the polymorphism at said marker results at least from a deletion and/or addition of nucleotide(s).

A pair of primers can be used for determining the presence of a given virulence marker.

In an advantageous embodiment, the same pair of primers can be used for determining the presence of a plurality of given virulence markers, e.g. by amplifying fragments with different size.

Markers of virulence and of presence of V. inaequalis are in particular as defined above.

### Step c)

During step c), if at least one virulence polymorphism is detected during step b), it is deduced therefrom that the sample provided during step a) comprises a virulent strain of Venturia inaequalis.

During step c), if a marker of presence of V. inaequalis is detected but none of the virulence polymorphisms are detected during step b), it is deduced therefrom that the sample provided during step a) does not comprise a virulent strain of Venturia inaequalis corresponding to the virulence polymorphisms tested.

If no DNA from a strain of Venturia inaequalis is detected in the sample, then the sample does not comprise any strain of Venturia inaequalis.

### Method for the Preventive and/or Curative Treatment of Apple Trees

A further subject matter of the present invention is a method for the preventive and/or curative treatment of apple trees comprising the resistance gene Rvi6, in particular allowing reducing the frequency and/or the quantity of phytosanitary product applied against Venturia inaequalis, comprising:
the implementation of steps a) to c) of the method as defined above for detecting the presence of a virulent strain of Venturia inaequalis in a sample, and
the application of a preventive and/or curative treatment if the sample provided during step a) comprises a virulent strain of Venturia inaequalis.

The method of detecting the presence of a virulent strain of Venturia inaequalis according to the invention can thus serve as a decision-making support tool for knowing whether the application of a preventive and/or curative treatment is necessary.

The preventive and/or curative treatment is preferentially carried out around the sample sampling area, e.g. in a nursery, orchard or on the farm around the sample sampling area where other trees carrying the resistance gene Rvi6 are planted.

The preventive and/or curative treatment comprises e.g. the application of an appropriate phytosanitary product against Venturia inaequalis, such as e.g. a fungicide.

The fungicide is chosen e.g. from a copper fungicide or a fungicide belonging to the strobilurine or anilinopyrimidine family.

### Method of Evaluating the Quality of an Apple Plant

A further subject matter of the present invention is a method for evaluating the quality of an apple plant, in particular an apple plant comprising a resistance gene Rvi6, in particular before the marketing thereof, comprising the following steps:
the implementation of steps a) to c) of the method as defined above for detecting the presence of a virulent strain of Venturia inaequalis in a sample, the sample provided during step a) being sampled from the air around said plant and/or from all or part of said plant, and
if no virulence polymorphism is detected during step b), deducing therefrom that the plant is not contaminated with a virulent Venturia inaequalis strain comprising the virulence polymorphism sought during step b).

In the above method, if (i) no allele comprising or consisting of sequences SEQ ID NO: 1, SEQ ID NO: 2, SEQ ID NO: 3, SEQ ID NO: 4, SEQ ID NO: 5 and SEQ ID NO: 6, and no promoter comprising or consisting of sequences SEQ ID NO: 7 and SEQ ID NO: 8 is detected during step a) and (ii) if DNA from a strain of Venturia inaequalis is detected in the sample, it is deduced therefrom that the plant is not contaminated with a virulent strain of Venturia inaequalis comprising virulence polymorphism.

### Geographical Mapping Method of the Virulence of Venturia inaequalis

A further subject matter of the present invention is a method of mapping the virulence of Venturia inaequalis, in particular against an apple tree comprising a resistance gene Rvi6, comprising the following steps:
implementing steps (a) to (c) of the method, as defined above, for detecting the presence of a virulent strain of Venturia inaequalis in a sample, from at least two samples sampled from different geographical areas,
deducing therefrom that the geographical area from which comes a sample comprising a virulent strain of Venturia inaequalis, is a virulence area and that the geographical area from which comes a sample comprising no virulent strain of Venturia inaequalis, is a virulence-free area.

The geographical area can be e.g. an orchard, a farm, a production basin, a region, a country, a continent and/or an island.

### Kit for the Detection of the Presence of a Virulent Strain of Venturia inaequalis

A further subject matter of the present invention is a kit for the detection of the presence of a virulent strain of Venturia inaequalis, in particular against an apple tree comprising a resistance gene Rvi6, in a sample, said kit comprising means for amplifying at least one virulence marker as defined above and, optionally, at least one marker for the presence of Venturia inaequalis as defined above.

The kit may, e.g., comprise means for amplifying the markers of the combination of at least two virulence markers and, optionally, at least one marker for the presence of V. inaequalis as defined above.

The means for amplifying a marker comprise e.g. at least one pair of primers specific for said marker(s) which are e.g. as defined hereinabove.

25

### Use of a Virulence Marker, of a Combination of Virulence Markers or of the Kit for the Detection of the Presence of a Virulent Strain of Venturia inaequalis

A further subject matter of the present invention is the use of a virulence marker as defined above, of a combination of at least two virulence markers and, optionally, of at least one marker for the presence of V. inaequalis as defined above, and/or a kit as defined above for detecting the presence of a virulent strain of Venturia inaequalis, in particular against an apple tree comprising a resistance gene Rvi6, or for determining whether a strain of Venturia inaequalis is virulent or avirulent, in particular against an apple tree comprising a resistance gene Rvi6.

The use as defined above makes it possible e.g. to determine geographical areas of virulence or avirulence, to evaluate whether a fungicidal treatment is needed, for evaluating the quality of an apple plant and/or apple fruits, e.g. before marketing in France or for export, and/or evaluating whether the virulence is present over a given territory so as to support the decision-making on whether or not to plant apple tree varieties carrying the resistance gene Rvi6.

Other features and advantages of the invention will become clearer from the following examples, which are given only as non-limiting illustration.

### SEQUENCE LISTING

SEQ ID NO: 1 is the sequence of an allele imparting a virulent character, as present in strain 2199.
SEQ ID NO: 2 is the sequence of an allele imparting a virulent character, as present in strain NL24.
SEQ ID NO: 3 is the sequence of an allele imparting a virulent character, as present in strain 1180.
SEQ ID NO: 4 is the sequence of an allele imparting a virulent character, as present in strain 2476.
SEQ ID NO: 5 is the sequence of an allele imparting a virulent character, as present in strain 1153.
SEQ ID NO: 6 is the sequence of an allele imparting a virulent character, as present in strain 08M205.
SEQ ID NO: 7 is an example of a sequence of the promoter of the gene AvrRvi6 imparting a virulent character, as present in strain 1286.
SEQ ID NO: 8 is an example of a sequence of the promoter of the gene AvrRvi6 imparting a virulent character, as present in strain 2207.
SEQ ID NO: 9 is the sequence of the housekeeping gene RPL4 of Venturia inaequalis.
SEQ ID NO: 10 and 11 correspond to the sequences of the forward and reverse primers, respectively, of a pair of primers used for the detection of the housekeeping gene RPL4 of Venturia inaequalis.
SEQ ID NO: 12 and 13 correspond to the sequences of the forward and reverse primers, respectively, of a pair of primers used for the detection of a virulent strain of Venturia inaequalis.
SEQ ID NO: 14 is an example of a sequence of the promoter of the gene AvrRvi6 imparting an avirulent character, as present in strain EUB04.
SEQ ID NO: 15 is the sequence of an allele imparting an avirulent character, as present in strain EUB04.

### FIGURES

FIG. 1 shows the results of quantitative PCR amplification of genomic DNA of the avirulent strain EUB04 at the locus AvRvi6, by using the primers SEQ ID NO: 12 and 13. The different curves correspond to a dilution range. The signal obtained in RFUs (relative fluorescence units) is given as a function of the number of PCR cycles.

FIG. 2 shows the results of the quantitative PCR amplification of the DNA of the virulent strain 2199 at the locus AvRvi6, by using the primers SEQ ID NO: 12 and 13. The different curves correspond to a dilution range. The signal obtained in RFUs (relative fluorescence units) is given as a function of the number of PCR cycles.

FIG. 3 shows the fusion curves of the amplicons of the locus AvRvi6 in the virulent strain 2199 and the avirulent strain EUB04, by using the primers SEQ ID NO: 12 and 13. The signal obtained in—d(RFU)/dT is given as a function of the temperature in Celsius.

### EXAMPLES

### Example 1: Polymorphism Analysis at Avirulence Locus AvrRvi6

Equipment and Methods

The avirulence Gene AvrRvi6, which interacts with the resistance gene Rvi6, was cloned, and the allelic polymorphism at the locus AvRvi6 and at the promoter thereof was analyzed on 150 strains of V. inaequalis coming from orchards and wild apple trees.

Results

The present analysis identified different polymorphisms associated with the virulent character and different polymorphisms associated with the avirulent character of Venturia inaequalis.

SEQ ID NO: 1 to 6 are examples of alleles conferring virulent character. SEQ ID NO: 7 and 8 are examples of the sequence of the AvrRvi6 promoter associated with a virulent character.

Such analysis of the polymorphism showed that virulent strains detected in European orchards have alleles at the locus AvRvi6, which are shared by Malus present in uncultivated spaces, thereby confirming that wild apple trees are the reservoirs of virulence responsible for such circumvention of resistance.

Moreover, the absence of observation of new cases of circumvention in orchards means that it is possible to develop a tool to specifically and exhaustively detect virulence against the resistance gene Rvi6 in apple orchards, from alleles identified within the virulent population present in the uncultivated section.

### Example 2: Method for Detecting the Presence of a Virulent Strain of Venturia inaequalis

Equipment and Methods

The genomic DNA of the strain to be tested is extracted from spores. The extraction is carried out according to the protocol of the NucleoSpin Food kit (Macherey Nagel).

The locus AvRvi6 is detected by quantitative PCR using the following reaction mixture for one reaction:
3.75 μl MesaBlue 2×(Eurogentec)
0.3 μl of forward primer at 10 μm (i.e. final concentration=200 Nm) of sequence SEQ ID NO: 12: CGATCCTTTTCGCAGTCT (Eurofins)
0.3 μl reverse primer to 10 μm of SEQ ID NO: 13: CTCAGTTGCATTGGCTTCGT (Eurofins)
3 μl dilute genomic DNA of Venturia inaequalis
Qsp 15 μl H₂O

The quantitative PCR program used is as follows (using a Biorad CFX connect apparatus):
1. 95° C. 5 minutes
2. 95° C. 3 seconds
3. 60° C. 1 minute
+reading the plate
4. Repeat steps 2 to 3, 39 times
5. Melting curve from 55° C. to 90° C. by step of 0.5° C./5s
+reading the plate

Results

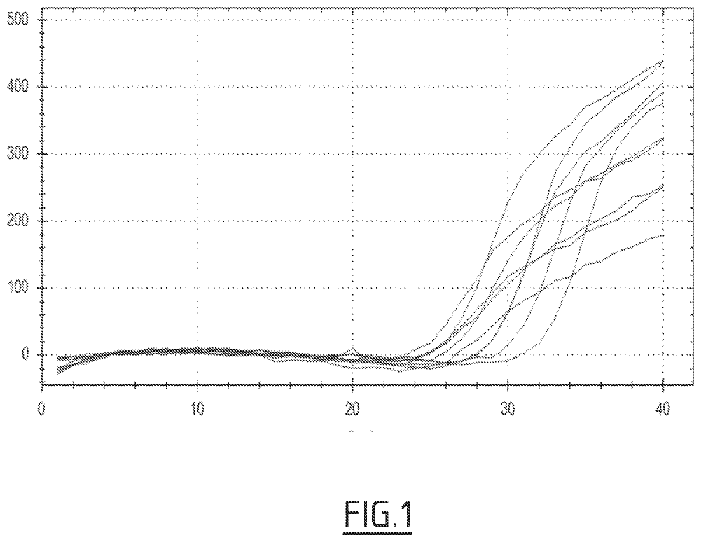

The results obtained are presented in FIGS. 1 to 3.

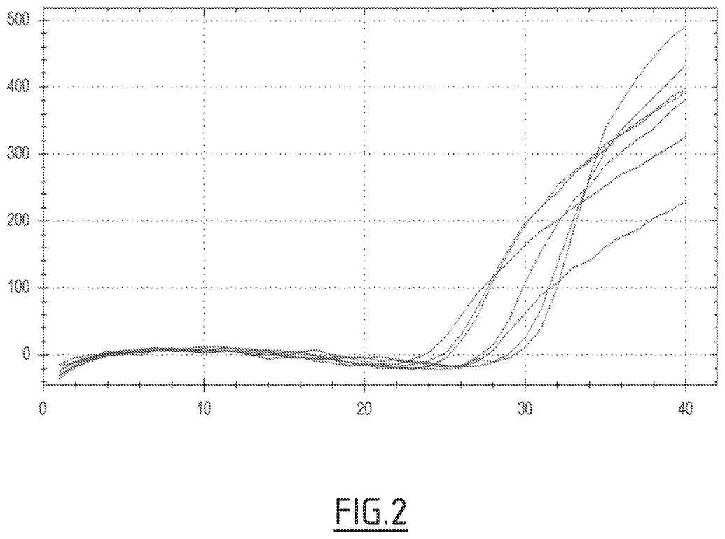

FIGS. 1 and 2 show that the method used amplifies the alleles of the avirulent strain B04 and of the virulent strain 2199, respectively, at the locus AvrRvi6.

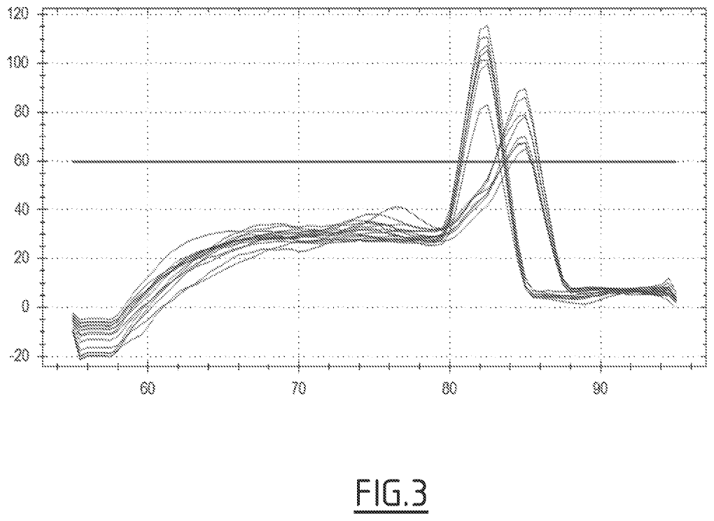

FIG. 3 shows that the melting curves of the two alleles are different, reflecting a deletion of nucleotides in the virulent strain compared with the avirulent strain. The virulence allele carried by strain 2199 is distinguished from the avirulence allele carried by strain B04 by a deletion of 155 pairs of bases.

The detection method according to the invention therefore can be used for detecting the presence of a virulent strain of Venturia inaequalis.

1. A method for detecting the presence of a virulent strain of Venturia inaequalis in a sample, comprising:
a) providing a sample likely to comprise Venturia inaequalis DNA,
b) determining whether the DNA present in the sample contains at least one virulence polymorphism,
c) If at least one virulence polymorphism is detected during step b), deducing therefrom that the sample provided during step a) comprises a virulent strain of Venturia inaequalis.

2. The detection method according to claim 1, characterized in that the virulence polymorphism is an allele comprising or consisting of a sequence selected from the group consisting of SEQ ID NO: 1, SEQ ID NO: 2, SEQ ID NO: 3, SEQ ID NO: 4, SEQ ID NO: 5 and SEQ ID NO: 6 or a promoter comprising or consisting of SEQ ID NO: 7 or SEQ ID NO: 8.

3. The method according to claim 1, characterized in that step b) comprises determining whether the DNA comprises at least one virulence marker selected from the group consisting of:
a fragment of sequence SEQ ID NO: 1 comprising nucleotides 155 to 165 of said sequence SEQ ID NO: 1,
a fragment of sequence SEQ ID NO: 2 comprising nucleotide 62 of said sequence SEQ ID NO: 2,
a fragment of sequence SEQ ID NO: 3 comprising nucleotide 160 of said sequence SEQ ID NO: 3,
a fragment of sequence SEQ ID NO: 4 comprising nucleotide 192 of said sequence SEQ ID NO: 4,
a fragment of sequence SEQ ID NO: 5 comprising nucleotide 201 of said sequence SEQ ID NO: 5,
a fragment of sequence SEQ ID NO: 6 comprising nucleotide 304 of said sequence SEQ ID NO: 6,
a fragment of sequence SEQ ID NO: 7 comprising nucleotide 189, nucleotides 304 to 316 and/or nucleotides 480 to 490 of said sequence SEQ ID NO: 7, and
a fragment of sequence SEQ ID NO: 8 comprising nucleotide 572 of said sequence SEQ ID NO: 8, nucleotide 706 of said sequence SEQ ID NO: 8, nucleotides 548 to 557 of said sequence SEQ ID NO: 8 and/or 15 consecutive nucleotides comprised between nucleotides 139 to 420 of said sequence SEQ ID NO: 8.

4. A method of preventive and/or curative treatment of apple trees comprising the resistance gene Rvi6 for reducing the frequency and/or amount of phytosanitary product applied against Venturia inaequalis, comprising:
implementing steps a) to c) of the method according to claim 1,
applying preventive and/or curative treatment if the sample provided during step a) includes a virulent strain of Venturia inaequalis.

5. A method of evaluating the quality of an apple plant, comprising the following steps:
implementing steps a) to c) of the method according to claim 1, the sample provided during step a) being sampled from the air around said plant and/or from all or part of said plant,
if no virulence polymorphism is detected during step b), deducing therefrom that the plant is not contaminated with a virulent Venturia inaequalis strain comprising the virulence polymorphism sought during step b).

6. A method of mapping the virulence of Venturia inaequalis comprising the following steps:
implementing steps a) to c) of the method according to claim 1, from at least two samples sampled from different geographical areas,
deducing therefrom that the geographical area from which comes a sample comprising a virulent strain of Venturia inaequalis, is a virulence area and that the geographical area from which comes a sample not comprising a virulent strain of Venturia inaequalis, is a virulence-free area.

7. A virulence marker comprising or consisting of:
a fragment of sequence SEQ ID NO: 1 comprising nucleotides 155 to 165 of said sequence SEQ ID NO: 1,
a fragment of sequence SEQ ID NO: 2 comprising nucleotide 62 of said sequence SEQ ID NO: 2,
a fragment of sequence SEQ ID NO: 3 comprising nucleotide 160 of said sequence SEQ ID NO: 3,
a fragment of sequence SEQ ID NO: 4 comprising nucleotide 192 of said sequence SEQ ID NO: 4,
a fragment of sequence SEQ ID NO: 5 comprising nucleotide 201 of said sequence SEQ ID NO: 5,
a fragment of sequence SEQ ID NO: 6 comprising nucleotide 304 of said sequence SEQ ID NO: 6,
a fragment of sequence SEQ ID NO: 7 comprising nucleotide 189, nucleotides 304 to 316 and/or nucleotides 480 to 490 of said sequence SEQ ID NO: 7, or
a fragment of sequence SEQ ID NO: 8 comprising nucleotide 572 of said sequence SEQ ID NO: 8, nucleotide 706, nucleotides 548 to 557 of said sequence SEQ ID NO: 8 and/or 15 consecutive nucleotides comprised between nucleotides 139 to 420 of said sequence SEQ ID NO: 8,
said fragment comprising at least 15 nucleotides.

8. A combination of at least two virulence markers, said virulence markers being as defined in claim 7.

9. A kit for detecting the presence of a virulent strain of Venturia inaequalis in a sample, said kit comprising means for amplifying the marker according to claim 7.

10. (canceled)

11. A kit for detecting the presence of a virulent strain of Venturia inaequalis in a sample, said kit comprising means for amplifying the markers of the combination according to claim 8.


SEQUENCE LISTING (15 sequences)

SEQ ID NO: 1 - DNA, 180 bp, Venturia inaequalis
atgcgtccccaaaccctcctcccagcgatccttttcgcacagtctagcttcgcatactcctgttgcttcgaagtgtacgatacccacagacaagtaatcgaaacaagatctaacaattgtatcataggctgggtcaaaaggatgtcgccaccggcagcaccacgaagccaatgcaactga

SEQ ID NO: 2 - DNA, 335 bp, Venturia inaequalis
atgcgtccccaaaccctcctcccagcgatccttttcgcacagtctagcttcgcatactcctcttgcttcgaagtgtacgatacccacagacaagtaatcgaaacaagatctaacaattgtatcataggctgggtcaaaaggatgtcgccaccggcgtatttgcgaacggcggtgtttttacatgggcaccacgcacggactgcataatcgaaatcaacaccaatgctgagagttgtcctgggtggcgttggagatatctctcggggacgtcttgtaagactttgggcttgcctctggcctatttaggtacagcaccacgaagccaatgcaactga

SEQ ID NO: 3 - DNA, 335 bp, Venturia inaequalis
atgcgtccccaaaccctcctcccagcgatccttttcgcacagtctagcttcgcatactcctgttgcttcgaagtgtacgatacccacagacaagtaatcgaaacaagatctaacaattgtatcataggctgggtcaaaaggatgtcgccaccggcgtatctgcgaacggcggtgtttttacatgggcaccacgcacggactgcataatcgaaatcaacaccaatgctgagagttgttctgggtggcgttggagatatctctcggggacgtcttgtaagactttgggcttgcctctggcctatttaggtacagcaccacgaagccaatgcaactga

SEQ ID NO: 4 - DNA, 335 bp, Venturia inaequalis
atgcgtccccaaaccctcctcccagcgatccttttcgcacagtctagcttcgcatactcctgttgcttcgaagtgtacgatacccacagacaagtaatcgaaacaagatctaacaattgtatcataggctgggtcaaaaggatgtcgccaccggcgtatttgcgaacggcggtgtttttacatgggcaccaagcacggactgcataatcgaaatcaacgccaatgctgagagttgttctgggtggcgttggagatatctctcggggacgtcttgtaagactttgggcttgcctctggcctatttaggtacagcaccacgaagccaatgcaactga

SEQ ID NO: 5 - DNA, 335 bp, Venturia inaequalis
atgcgtccccaaaccctcctcccagcgatccttttcgcacagtctagcttcgcatactcctgttgcttcgaagtgtacgatacccacagacaagtaatcgaaacaagatctaacaattgtatcataggctgggtcaaaaggatgtcgccaccggcgtatttgcgaacggcggtgtttttacatgggcaccacgcacggaccgcataatcgaaatcaacaccaatgctgagagttgttctgggtggcgttggagatatctctcggggacgtcttgtaagactttgggcttgcctctggcctatttaggtacagcaccacgaagccaatgcaactga

SEQ ID NO: 6 - DNA, 335 bp, Venturia inaequalis
atgcgtccccaaaccctcctcccagcgatccttttcgcacagtctagcttcgcatactcctgttgcttcgaagtgtacgatacccacagacaagtaatcgaaacaagatctaacaattgtatcataggctgggtcaaagggatgtcgccaccggcgtatttgcgaacggcggtgtttttacatgggcaccacgcacggactgcataatcgaaatcaacaccaatgctgagagttgttctgggtggcgttggagatatctctcggggacgtcttgtaagtctttgggcttgcctctggcctattgaggtacagcaccacgaagccaatgcaactga

SEQ ID NO: 7 - DNA, 502 bp, Venturia inaequalis
tactctacaccggctgcacgcgtagctccaacaacagctgggtattcgccccaccagtttctctctatttagtggagatttcttgtctattttagaagagatatattacttacggatcagcgaacctccatctactgttaaagttacctatattaaggtggcttagtatccctacgtctctctggagtatcctttccaagtccttggctattatcaatgcttcaatccttggccttcagaatcttacggcggctttaaatatacacatgaagatgcagagtgtccggaccaaggccgaacccacaatatagaatagttgcccatgtctatgttaccaaaaatagttgaactcatttatcggacagaggatctagtccgatcatatccgcagccttgtactccgtatctatttagactcgagtttcccacctcttcctgccttcattatcactgcaaccatcatcaatacttctatcaatagaatactcgaaaaaagccaaca

SEQ ID NO: 8 - DNA, 745 bp, Venturia inaequalis
acagctgggtattcgccccaccagtttctctctatttagtggagatttcttgtctattttagaagagatatattacttacggatcagcgaacctccatctactgttaaagttacctatattaaggtggcttagtatccctacggaggtctgaatgattgaaattaagcgttttcaaaagcgggccccgctaaactattagactggtatggatttcttacaagattaccgttccaggggctacggctaaattatagaggatacctgctatatctcctgggtaccttagggggatcgcccggactttgattctttacaccatagaatacgagtctaaacctatatagtctgagagtagagggcccgagcgattagtggctgagaaaagtggaaacgcttactttcaatcgttcaaacctccgtacgtctctctggagtgtcctttccaagtccttggctattatcaatgcttcaatccttggccttcagaatcttacggcggctttaaatatacacatgaagatgcagagtgtccggaccaaggccgaacccacaatatagaatagttgcacatgtctatgttaccaaaaatagttgaactcatttatcggacagaggatctagtccgatcatatccgcagccttgtactccgtatctatttagactcgagtttcccacctcttcctgccttcattatcactgcgaccatcatcaatacttctatcaatactcgaaaaaagccaaca

SEQ ID NO: 9 - DNA, 1454 bp, Venturia inaequalis
atgctgagctggtcaggtgactcaatcatatccctgccagcttcgccggcgttgattggatgattcagcccactcgaaattcgtgtgccgcgatttttcgaacctttccatcgtcgtccccagtcttaggcctttccaacaacaaactaatccagcagcaacaatgacctccagaccgactgttggcatcgtcggcgccgatggcgttgccacgaagaacacgattccactcccaagtgtcttcaaggccccgatccgaccagatattgtccagtatgcgactctaccatcacacggcacaaaatcgaaatgctgacggaaagacagaactgtccacactggcatggccaagaacaagaggcagccatatgctgtgagcgagaaggccggtcaccagacctctgccgagtcctggggtactggtgagtgtaattggacatttggagctttggctccaaaatggattgatagatatgataaattctgacaatacaattacaggacgtgccgttgcgcgtattcctcgtgtctctggtggtggtacccaccgcgctggtcaagccgcctttggtaacatgtgccgatccggtcgcatgttcgcacccacgaaggtctggaggaagtggcacgtcaagatcaaccttggacagaagtgagttgagagtcttcgaaatgtatggtcgagtctaacaaatcccaggcgattcgctaccgcctctgccattgctgcctcatccaacgcagctctcctcatggcccgtggtcaccgtgtttccaccgttccagaggtcccactcgttgtctcctccgccgcattcaacgtcacaaagacatccgctgctgtcgctctcctcaagggaattggtgctggcccagatgttgacaaggtcaagaagtcgagaaagcttcgcgctggcaagggtaagctcagaggccgccgtcaccgtcaacgacgtggacctctcgtagtctacaacccagataccgatggcaaggagatctaccagtcgttccggaacatcgccggtgtcgagacgtcctcagtctactctctcaacctgctccagctcgcaccaggtggacatcttggacgattcattgtttggacttccggtgctttcgaggcgctcgacaagatctacggatctaccgtcgacccatctgcgctcaagaaggacttcctcctcccatccccaaccgtcgcccagccagatatcgccaagctcatcaattcctcggaaattcaatctgctctccgaggtgtccgtggcagcaacattaccaagcgtggtgttacacaaaagaagaaccctctcctcaataagcagattcttctacgcctcaacccatacgcgtcgcaatacgccaaggagaagttgggtcagcaaaaggtcgagggcgagaagccaaaggcattagatgctagcttcttgaagcttgctcacgaaaactaa

SEQ ID NO: 10 - DNA, 20 bp, Artificial Sequence
  amorce sens RPL4
ttcaatctgctctccgaggt

SEQ ID NO: 11 - DNA, 20 bp, Artificial Sequence
  amorce antisens RPL4
ctaatgcctttggcttctcg

SEQ ID NO: 12 - DNA, 20 bp, Artificial Sequence
  amorce sens
cgatccttttcgcacagtct

SEQ ID NO: 13 - DNA, 20 bp, Artificial Sequence
  amorce antisens
ctcagttgcattggcttcgt

SEQ ID NO: 14 - DNA, 745 bp, Venturia inaequalis
caccttaagtgctaaggccgtacaatatcgcttgtaagctatattacacctcttaccttccagtctaacatttgtgtagggacaaaattgatagctatttagatattctacagcacggcttcattggcttcattgccaagtataatattgagctaactactttaggtgcggaaagaagccctcaacgctatctcggccaaatgtaagacccctggaccgaagttcactttcgaaggttaatgcggtactctacaccggctgcacgcgtagctccaacaacagctgggtattcgccccaccagtttctctctatttagtggagatttcttgtctattttagaagagatatattacttacggatcagcgaacctccatctactgttaaagttacctatattaaggtggcttagtatccctacgtctctctggagtgtcctttccaagtccttggctattatcaatgcttcaatccttggccttcagaatcttacggcggctttaaatatacacatgaagatgcagagtgtccggaccaaggccgaacccacaataatatagaatagttgcccatgtctatgttaccaaaaatagttgaactcatttatcggacagaggatctagtccgatcatatccgcagccttgtactccgtatctatttagactcgagtttcccacctcttcctgccttcattatcactgcaaccatcatcaatacttctatcaatactcgaaaaaagccaaca

SEQ ID NO: 15 - DNA, 335 bp, Venturia inaequalis
atgcgtccccaaaccctcctcccagcgatccttttcgcacagtctagcttcgcatactcctgttgcttcgaagtgtacgatacccacagacaagtaatcgaaacaagatctaacaattgtatcataggctgggtcaaagggatgtcgccaccggcgtatttgcgaacggcggtgtttttacatgggcaccacgcacggactgcataatcgaaatcaacaccaatgctgagagttgttctgggtggcgttggagatatctctcggggacgtcttgtaagtctttgggcttgcctctggcctatttaggtacagcaccacgaagccaatgcaactga